SEQUENCE LISTING (22 sequences)

SEQ ID NO: 1 - protein, 200 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHP

SEQ ID NO: 2 - protein, 437 aa, synthetic construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 437 aa, synthetic construct
LFTVTVPKELYIIEHGSNVTLECNFDTGSHVNLGAITASLQKVENDTSPHRERATLLEEQLPLGKASFHIPQVQVRDEGQYQCIIIYDDDWLYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 437 aa, synthetic construct
LFTVTVPKELYIIEHGSNVTLECNFDTGSHVNLGAITASLQKVENDTSPHRERATLLEEQLPLGKASFHIPQVQVRDEGQYQCIIDYDDDDLYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 437 aa, synthetic construct
LFTVTVPKELYIIEHGSNVTLECNFDTGSHVNLGAITASLQKVENDTSPHRERATLLEEQLPLGKASFHIPQVQVRDEGQYLCDIIYGVAWDDKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 437 aa, synthetic construct
LFTVTVPKELYIIEHGSNVTLECNFDTGSHVNLGAITASLQKVENDTSPHRERATLLEEQLPLGKASFHIPQVQVRDEGQYLCDIDYGVADDDKDLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 437 aa, synthetic construct
IDTVTVPKELYIIEHGSNVTLECNFDTGSHVNLGAITASLQKVENDTSPHRERATLLEEQLPLGKASFHIPQVQVRDEGQYQCIIIYGVDWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8-10 - skipped (3 entries)

SEQ ID NO: 11 - protein, 15 aa, synthetic construct
TSESFVLNWYRMSPS

SEQ ID NO: 12 - protein, 15 aa, synthetic construct
FSNTSESFVLNWYRM

SEQ ID NO: 13 - protein, 12 aa, synthetic construct
ESFVLNWYRMSP

SEQ ID NO: 14 - protein, 15 aa, synthetic construct
TYLCGAISLAPKAQI

SEQ ID NO: 15-20 - skipped (6 entries)

SEQ ID NO: 21 - protein, 14 aa, Homo sapiens
  VARIANT 2: note = Any amino acid
  VARIANT 4: note = Any amino acid
  VARIANT 6: note = Any amino acid
  VARIANT 7: note = Any amino acid
  VARIANT 8: note = Any amino acid
  VARIANT 9: note = Any amino acid
  VARIANT 11: note = Any amino acid
  VARIANT 13: note = Any amino acid
QXIXIXXXXWXYXY

SEQ ID NO: 22 - protein, 7 aa, Homo sapiens
  VARIANT 2: note = Any amino acid
IXGVAWD

(19) United States
(12) Patent Application Publication
(10) Pub. No.: US 2025/0051419 A1
(43) Pub. Date: Feb. 13, 2025

(54) POLYPEPTIDE MODULATORS

(71) Applicant: Georgiamune Inc., Gaithersburg, MD (US)

(72) Inventors: SAMIR KHLEIF, Bethesda, MD (US); Mikayel MKRTICHYAN, Tujunga, CA (US)

(21) Appl. No.: 18/718,397

(22) PCT Filed: Dec. 8, 2022
(86) PCT No.: PCT/US2022/081186

§ 371 Date: Jun. 10, 2024

Related U.S. Application Data
(60) Provisional application No. 63/288,330, filed on Dec. 10, 2021.

(51) Int. Cl. C07K 14/705; A61K 38/00; A61K 45/06

(52) U.S. Cl. CPC C07K 14/70532; A61K 45/06; A61K 38/00; C07K 2319/30

(57) ABSTRACT

Isolated polypeptides are described, which are mutants of programmed death 1 ligand 2 (“PD-L2”) and can either activate or suppress immune cells through activating or suppressing programmed cell death protein 1 (“PD-1”). Also described are isolated polypeptides that block the activating or inhibitory site of PD-L2, thereby inhibiting or activating PD-1. Further described are pharmaceutical compositions including the polypeptides and methods of using the polypeptides.

### CROSS-REFERENCE TO RELATED APPLICATIONS

This application claims the benefit and priority of U.S. Provisional Application No. 63/288,330, filed on Dec. 10, 2021, the content of which is incorporated herein by reference in its entirety.

### INCORPORATION BY REFERENCE

Any patent, patent publication, journal publication, or other document cited herein is expressly incorporated herein by reference in its entirety.

### FIELD OF THE INVENTION

This invention is generally related to polypeptide modulators and immunomodulation. More specifically, this invention is related to ligands that specifically bind to or modulate programmed cell death protein 1 (“PD-1”), and methods of use thereof.

### BACKGROUND OF THE INVENTION

PD-1 is a protein on the surface of T and B cells that has a role in regulating the immune system's response to the cells of the human body by down-regulating the immune system and promoting self-tolerance by suppressing T cell inflammatory activity. This biological pathway prevents autoimmune diseases, but it can also prevent the immune system from killing cancer cells.

Programmed death-ligand 1 (“PD-L1”) binds to PD-1 and transmits an inhibitory signal to suppress the immune response. The PD-1/PD-L1 pathway has shown promising clinical success as a cancer immunotherapy target. Current antibodies that target either PD-1 or PD-L1 can block this interaction and boost immune response against cancer cells. Successful clinical trials with PD-1 monoclonal antibodies and other immune-checkpoint inhibitors have opened new avenues in cancer immunology. However, the failure of a large subset of cancer patients to respond to new immunotherapies has led to intensified research on combination therapies and predictive biomarkers. See, e.g., Iwai, Y, et al., Journal of Biomedical Science, 24:26 (2017).

Besides PD-L1, programmed cell death 1 ligand 2 (“PD-L2”) is another protein known to bind to PD-1.

Thus, it is an object of one or more inventions disclosed herein to provide compositions and methods for modulating PD-1 signal transduction and related biological pathways.

### SUMMARY OF THE INVENTION

In one aspect, isolated polypeptides that bind to PD-1 are described. In certain embodiments, the polypeptide can modulate an immune response in vitro or in vivo, and/or can also be used, individually or in combination with other agents, in the prevention or treatment of a variety of conditions. In another aspect, isolated polypeptides that block the inhibitory or activating site of PD-L2 are described. In yet another aspect, pharmaceutical compositions including the polypeptides and methods of using these compositions, individually or in combination with other agents or compositions, in the prevention or treatment of a variety of conditions are described. In yet another aspect, inducible T cell kinase (“ITK”) activators or inhibitors are described, and/or can also be used, individually or in combination with other agents, in the prevention or treatment of a variety of conditions.

In one aspect, an isolated polypeptide is described, including an amino acid sequence which is at least 60%, 65%, 70%, 75%, 80%, 85%, 90%, 95%, or 99% identical to SEQ ID NO: 1.

In another aspect, an isolated polypeptide is described, including an amino acid sequence which is SEQ ID NO: 21.

In yet another aspect, an isolated polypeptide is described, including an amino acid sequence which is SEQ ID NO: 22.

In any one of the embodiments disclosed herein, the amino acid is fused to an immunoglobulin.

In any one of the embodiments disclosed herein, the immunoglobulin is IgG1, IgG2, IgG3, or IgG4.

In any one of the embodiments disclosed herein, the polypeptide includes an amino acid sequence which is at least 60%, 65%, 70%, 75%, 80%, 85%, 90%, 95%, or 99% identical to SEQ ID NO: 3 or SEQ ID NO: 4.

In any one of the embodiments disclosed herein, the polypeptide includes an amino acid sequence which is SEQ ID NO: 3 or SEQ ID NO: 4.

In any one of the embodiments disclosed herein, the polypeptide includes one or more amino acids each selected from the group consisting of Tyr 112, Trp 110, Ile 103, Ile 105, Gln 101, and Tyr 114.

In any one of the embodiments disclosed herein, the polypeptide binds to PD-1 and activates PD-1.

In any one of the embodiments disclosed herein, the polypeptide activates immune cells.

In any one of the embodiments disclosed herein, the polypeptide includes an amino acid sequence which is at least 60%, 65%, 70%, 75%, 80%, 85%, 90%, 95%, or 99% identical to SEQ ID NO: 5 or SEQ ID NO: 6.

In any one of the embodiments disclosed herein, the polypeptide includes an amino acid sequence which is SEQ ID NO: 5 or SEQ ID NO: 6.

In any one of the embodiments disclosed herein, the polypeptide includes one or more amino acids each selected from the group consisting of Ile 105, Val 108, Gly 107, Ala 109, Trp 110, and Asp 111.

In any one of the embodiments disclosed herein, the polypeptide binds to PD-1 and inhibits PD-1.

In any one of the embodiments disclosed herein, the polypeptide suppresses immune cells.

In any one of the embodiments disclosed herein, the polypeptide induces central memory T cells (“Tcm”).

In any one of the embodiments disclosed herein, the polypeptide prevents T cell exhaustion.

In yet another aspect, a conjugate is described, including the polypeptide of any one of the embodiments disclosed herein, where the polypeptide is attached to a detectable marker or a carrier molecule.

In any one of the embodiments disclosed herein, the carrier molecule is selected from the group consisting of glycosaminoglycan, proteoglycan, albumin, and polyalkylene glycol.

In yet another aspect, a nucleic acid is described, encoding the polypeptide of any one of the embodiments disclosed herein.

In yet another aspect, a pharmaceutical composition is described, including the polypeptide of any one of the embodiments disclosed herein.

In any one of the embodiments disclosed herein, the polypeptide is encapsulated in a liposome.

In any one of the embodiments disclosed herein, the pharmaceutical composition further includes a second therapeutic agent.

In any one of the embodiments disclosed herein, the second therapeutic agent is a chemotherapeutic agent or an immunosuppressive agent.

In yet another aspect, a method of inducing, promoting, or enhancing an immune response in a subject in need thereof is described, including administering to the subject an effective amount of the polypeptide of any one of the embodiments disclosed herein, the conjugate of any one of the embodiments disclosed herein, or the pharmaceutical composition of any one of the embodiments disclosed herein.

In yet another aspect, a method of treating cancer or reducing tumor burden in a subject in need thereof is described, including administering to the subject an effective amount of the polypeptide of any one of the embodiments disclosed herein, the conjugate of any one of the embodiments disclosed herein, or the pharmaceutical composition of any one of the embodiments disclosed herein.

In any one of the embodiments disclosed herein, the cancer is selected from the group consisting of adult T-cell leukemia/lymphoma, bladder, brain, breast, cervical, colorectal, esophageal, kidney, liver, lung, nasopharyngeal, pancreatic, prostate, skin, stomach, uterine, ovarian, and testicular cancer.

In any one of the embodiments disclosed herein, the method further includes upregulating ITK.

In yet another aspect, a method of reducing, suppressing, or preventing an immune response in a subject in need thereof is described, including administering to the subject an effective amount of the polypeptide of any one of the embodiments disclosed herein, the conjugate of any one of the embodiments disclosed herein, or the pharmaceutical composition of any one of the embodiments disclosed herein.

In yet another aspect, a method of treating an autoimmune disease in a subject in need thereof is described, including administering to the subject an effective amount of the polypeptide of any one of the embodiments disclosed herein, the conjugate of any one of the embodiments disclosed herein, or the pharmaceutical composition of any one of the embodiments disclosed herein.

In any one of the embodiments disclosed herein, the autoimmune disease is selected from the group consisting of achalasia, Addison's disease, adult Still's disease, agammaglobulinemia, alopecia areata, amyloidosis, ankylosing spondylitis, anti-glomerular basement membrane disease, anti-tubular basement membrane antibody nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, autoimmune urticaria, axonal and neuronal neuropathy, Baló disease, Behcet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss syndrome, eosinophilic granulomatosis, cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura, pemphigoid gestationis, hidradenitis suppurativa (acne inversa), hypogammaglobulinemia, IgA nephropathy, IgG4-related sclerosing disease, immune thrombocytopenia purpura, inclusion body myositis, interstitial cystitis, juvenile arthritis, juvenile diabetes (type 1 diabetes), juvenile myositis, Kawasaki disease, Lambert-Eaton syndrome, leukocytoclastic vasculitis, lichen planus, lichen sclerosus, ligneous conjunctivitis, linear IgA disease, lupus, chronic Lyme disease, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, pediatric autoimmune neuropsychiatric disorder, paraneoplastic cerebellar degeneration, paroxysmal nocturnal hemoglobinuria, Parry Romberg syndrome, pars planitis (peripheral uveitis), Parsonage-Turner syndrome, pemphigus, peripheral neuropathy, perivenous encephalomyelitis, pernicious anemia, POEMS syndrome, polyarteritis nodosa, polyglandular syndrome type I, polyglandular syndrome type II, polyglandular syndrome type III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome, subacute bacterial endocarditis, Susac's syndrome, sympathetic ophthalmia, Takayasu's arteritis, temporal arteritis (giant cell arteritis), thrombocytopenia purpura, Tolosa-Hunt syndrome, transverse myelitis, ulcerative colitis, undifferentiated connective tissue disease, uveitis, vasculitis, vitiligo, Vogt-Koyanagi-Harada disease, and a combination thereof.

In any one of the embodiments disclosed herein, the method further includes downregulating ITK.

In yet another aspect, a method of inducing, promoting, or enhancing an immune response in a subject in need thereof is described, including administering to the subject an effective amount of an activator of ITK.

In yet another aspect, a method of treating cancer or reducing tumor burden in a subject in need thereof is described, including administering to the subject an effective amount of an activator of ITK.

In any one of the embodiments disclosed herein, the activator of ITK is a small-molecule compound, a polypeptide, or a nucleic acid.

In yet another aspect, a method of reducing, suppressing, or preventing an immune response in a subject in need thereof is described, including administering to the subject an effective amount of an inhibitor of ITK.

In yet another aspect, a method of treating an autoimmune disease in a subject in need thereof is described, including administering to the subject an effective amount of an inhibitor of ITK.

In any one of the embodiments disclosed herein, the inhibitor of ITK is a small-molecule compound, a polypeptide, or a nucleic acid.

In yet another aspect, an isolated polypeptide is described, including an amino acid sequence which is at least 60%, 65%, 70%, 75%, 80%, 85%, 90%, 95%, or 99% identical to any one of SEQ ID NOS: 11-13.

In any one of the embodiments disclosed herein, the polypeptide includes an amino acid sequence selected from the group consisting of SEQ ID NOS: 11-13.

In any one of the embodiments disclosed herein, the polypeptide binds to PD-L2.

In any one of the embodiments disclosed herein, the polypeptide blocks the inhibitory site of PD-L2.

In yet another aspect, an isolated polypeptide is described, including an amino acid sequence which is at least 60%, 65%, 70%, 75%, 80%, 85%, 90%, 95%, or 99% identical to SEQ ID NO: 14.

In any one of the embodiments disclosed herein, the polypeptide includes an amino acid sequence which is SEQ ID NO: 14.

In any one of the embodiments disclosed herein, the polypeptide blocks the activating site of PD-L2.

In any one of the embodiments disclosed herein, the polypeptide inhibits immune cells.

In yet another aspect, a method of reducing, suppressing, or preventing an immune response in a subject in need thereof is described, including administering to the subject an effective amount of a small molecule that binds to an inhibitory site of PD-1.

In yet another aspect, a method of treating an autoimmune disease in a subject in need thereof is described, including administering to the subject an effective amount of a small molecule that binds to an inhibitory site of PD-1.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### BRIEF DESCRIPTION OF THE DRAWINGS

FIG. 1 shows the relative luminescence units (“RLU”) of the groups of PD-1 reporter cells treated in the presence or absence of a polypeptide, according to one or more embodiments described herein.

FIGS. 2A-2C show the design rationale of polypeptides that bind to PD-L2, according to one or more embodiments described herein.

FIGS. 3A-3B show the design rationale of PD-L2 mutants, according to one or more embodiments described herein.

FIG. 4 shows that the type of PD-1 signal depends on the balance between ITK and Src homology region 2 (“SH2”)-containing protein tyrosine phosphatase 2 (“SHP2”), according to one or more embodiments described herein.

FIGS. 5A-5C show the immune response dictated by the balance between ITK and SHP2 and the upstream pathways affecting the balance between ITK and SHP2, according to one or more embodiments described herein.

FIGS. 6A-6C show differences in the levels of PD-1-bound ITK and SHP2 after treatment of primary T cells with PD-L1 and PD-L2, according to one or more embodiments described herein.

FIGS. 7A-7C show that PD-L2 increases Tcm and prevents T cell exhaustion, according to one or more embodiments described herein.

FIGS. 8A-8C show that PD-L2 is required for Tcm generation and prevention of exhaustion, according to one or more embodiments described herein.

FIGS. 9A-9D show that PD-L2 is required for initiation of activation of both CD4 and CD8 T cells, according to one or more embodiments described herein.

### DETAILED DESCRIPTION OF THE INVENTION

### Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting.

Unless defined otherwise, all technical and scientific terms used herein have the same meaning as commonly understood by one of ordinary skill in the art to which this disclosure belongs. Any compositions, methods, and materials similar or equivalent to those described herein can be used in the practice or testing of the present invention.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately ±10%. In some embodiments, the values may be either above or below the stated value in a range of approximately ±5%. In some embodiments, the values may be either above or below the stated value in a range of approximately ±2%. In some embodiments, the values may be either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “exemplary”, “such as”, “for example”, “including, but not limited to”) provided herein, is intended merely to better illuminate the invention and does not pose a limitation on the scope of the invention unless otherwise indicated.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, adult T-cell leukemia/lymphoma (including that caused by human T-cell lymphotropic virus (“HTLV-1”)), biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. As used herein, the term “lymphoma” refers to cancer of the lymphatic system or a blood cancer that develops from lymphocytes. Cancers can be primary or metastatic. Diseases other than cancers may be associated with mutational alternation of component of Ras signaling pathways and the polypeptide disclosed herein may be used to treat these non-cancer diseases. Such non-cancer diseases include, but are not limited to, neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arteriovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

The term “immune cell” as used herein refers to cells of the innate and acquired immune system including, but not limited to, neutrophils, eosinophils, basophils, glial cells (e.g., astrocytes, microglia, and oligodendrocytes), monocytes, macrophages, dendritic cells, and lymphocytes (e.g., B cells, T cells, and natural killer (“NK”) cells).

As used herein, “conventional T cells” are T lymphocytes that express an αβ T cell receptor (“TCR”) as well as a co-receptor, such as CD4 or CD8. Conventional T cells are present in the peripheral blood, lymph nodes, and tissues. See Roberts and Girardi, “Conventional and Unconventional T Cells”, Clinical and Basic Immunodermatology, pp. 85-104, (Gaspari and Tyring (ed.)), Springer London (2008), herein incorporated by reference in its entirety. As used herein, “unconventional T cells” are lymphocytes that express a γδ TCR and may commonly reside in an epithelial environment, such as the skin, gastrointestinal tract, or genitourinary tract. Another subset of unconventional T cells is the invariant natural killer T (“NKT”) cell, which has phenotypic and functional capacities of a conventional T cell, as well as features of natural killer cells (e.g., cytolytic activity). See id. As used herein, regulatory T cells (“Tregs”) are a subpopulation of T cells which modulate the immune system, maintain tolerance to self-antigens, abrogate autoimmune disease, and otherwise suppress immune-stimulating or activating responses of other cells. Tregs come in many forms, with the most well-understood being those that express CD4, CD25, and Foxp3. As used herein, “natural Treg” or “nTreg” refer to a Treg or cells that develop in the thymus. As used herein, “induced Treg” or “iTreg” refer to a Treg or cells that develop from mature CD4+ conventional T cells outside of the thymus.

As used herein, the term “peptide” or “polypeptide” refers to a chain of amino acids of any length, regardless of modification (e.g., phosphorylation or glycosylation). The terms “peptide” and “polypeptide” are used interchangeably. The terms include proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: alanine (Ala, A), arginine (Arg, R), asparagine (Asn, N), aspartic Acid (Asp, D), cysteine (Cys, C), glutamine (Gln, Q), glutamic Acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

The term “immune response” includes, for example, the development of a beneficial humoral (antibody-mediated) and/or a cellular (mediated by antigen-specific T cells or their secretion products) response directed against a polypeptide in a recipient patient. Such a response can be an active response, induced by administration of immunogen, or a passive response, induced by administration of antibody or primed T cells. A cellular immune response is elicited by the presentation of polypeptide epitopes in association with class I or class II major histocompatibility complex (“MHC”) molecules to activate antigen-specific CD4+ T helper cells and/or CD8+ cytotoxic T cells. The response can also involve activation of monocytes, macrophages, NK cells, basophils, dendritic cells, astrocytes, microglia cells, eosinophils, activation or recruitment of neutrophils, or other components of innate immunity. The presence of a cell-mediated immunological response can be determined by proliferation assays (CD4+ T cells) or cytotoxic T lymphocyte (“CTL”) assays. The relative contributions of humoral and cellular responses to the protective or therapeutic effect of an immunogen can be distinguished by separately isolating antibodies and T cells from an immunized syngeneic animal and measuring protective or therapeutic effect in a second subject.

The terms “immunogenic agent” or “immunogen” refer to an agent capable of inducing an immunological response against itself on administration to a mammal, optionally in conjunction with an adjuvant.

### Polypeptides

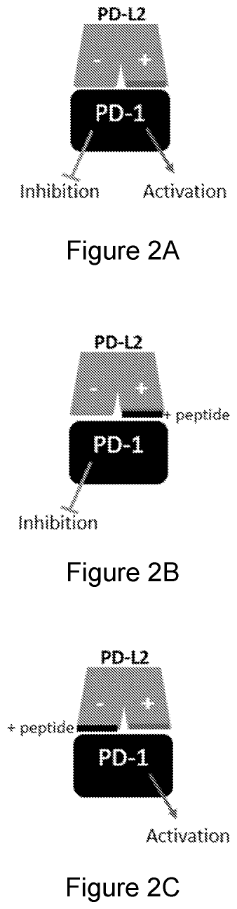

In addition to PD-L1, PD-L2 is another protein that binds to PD-1. Applicants have surprisingly found that when PD-L2 binds to PD-1 via two main interactions sites on PD-L2, these sites have opposite functions: one site on PD-L2 activates PD-1 and immune response while the other one inhibits PD-1 and immune response. The site that activates PD-1 is referred to herein as the “activating site” and the site that inhibits PD-1 is referred to herein as the “inhibitory site.” The two interaction sites of PD-L2 are illustrated in FIG. 2A. In some embodiments, the activating site on PD-L2 includes one or more amino acids each selected from the group consisting of Tyr 112, Trp 110, Ile 103, Ile 105, Gln 101, and Tyr 114. In some embodiments, the inhibitory site on PD-L2 includes one or more amino acids each selected from the group consisting of Ile 105, Val 108, Gly 107, Ala 109, Trp 110, and Asp 111.

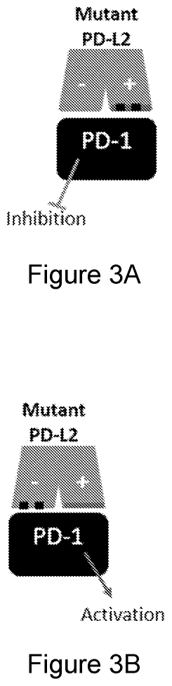

In some embodiments, a polypeptide blocking or disabling the inhibitory site of PD-L2 is described, which activates PD-1 and immune response. In other embodiments, a polypeptide blocking or disabling the activating site of PD-L2 is described, which inhibits PD-1 and suppresses the immune response. For example, as illustrated in FIG. 3B, a mutant of PD-L2 with a disabled inhibitory site could activate immune response through PD-1 signal transduction. Furthermore, as illustrated in FIG. 3A, a mutant of PD-L2 with a disabled activating site could suppress the immune response through PD-1 signal transduction.

SEQ ID NO: 1 is the amino acid sequence defining human PD-L2:

LFTVTVPKELYIIEHGSNVTLECNFDTGSHVNLGAITASLQKVENDTSPH
RERATLLEEQLPLGKASFHIPQVQVRDEGQYQCIIIYGVAWDYKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTH
P.

SEQ ID NO: 21 is the amino acid sequence defining the activating site of human PD-L2:

QXIXIXXXXWXYXY,

where X is any amino acid.

SEQ ID NO: 22 is the amino acid sequence defining the inhibitory site of human PD-L2:

IXGVAWD,

where X is any amino acid.

SEQ ID NO: 2 is the amino acid sequence defining human PD-L2 fused with hIgG1:

LFTVTVPKELYIIEHGSNVTLECNFDTGSHVNLGAITASLQKVENDTSPH
RERATLLEEQLPLGKASFHIPQVQVRDEGQYQCIIIYGVAWDYKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
IEGRMDPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TVDKSRWQQGNVFSCSVMHEALHNHYTQKSLSLSPGK.

In some embodiments, an isolated polypeptide or mutant thereof which is at least 60%, 65%, 70%, 75%, 80%, 85%, 90%, 95%, or 99% identical to SEQ ID NO: 1 is described. In some embodiments, the polypeptide is a mutant of SEQ ID NO: 1. In some embodiments, the polypeptide blocking or disabling the activating site of PD-L2, resulting in the inhibition of PD-1 and suppression of the immune response. In some embodiment, the polypeptide includes an amino acid sequence which is SEQ ID NO: 21. In some embodiment, the polypeptide includes an amino acid sequence which is SEQ ID NO: 22.

In some embodiments, the polypeptide includes an amino acid sequence which is any one of SEQ ID NOS: 1, 21, and 22; and the amino acid sequence is fused to an immunoglobulin or fragment thereof. In some embodiments, the immunoglobulin is IgG, IgM, or IgA. In some embodiments, the immunoglobulin is IgG1, IgG2, IgG3, or IgG4. In some embodiments, the immunoglobulin is human immunoglobulin. In some embodiments, the polypeptide has an amino acid sequence which is at least 60%, 65%, 70%, 75%, 80%, 85%, 90%, 95%, or 99% identical to SEQ ID NO: 2 is described. In some embodiments, the polypeptide is a mutant of SEQ ID NO: 2.

SEQ ID NO: 3 is the amino acid sequence defining a first mutant of PD-L2:

LFTVTVPKELYIIEHGSNVTLECNFDTGSHVNLGAITASLQKVENDTSPH
RERATLLEEQLPLGKASFHIPQVQVRDEGQYQCIIIYDDDWLYKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
IEGRMDPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TVDKSRWQQGNVFSCSVMHEALHNHYTQKSLSLSPGK.

SEQ ID NO: 4 is the amino acid sequence defining a second mutant of PD-L2:

LFTVTVPKELYIIEHGSNVTLECNFDTGSHVNLGAITASLQKVENDTSPH
RERATLLEEQLPLGKASFHIPQVQVRDEGQYQCIIDYDDDDLYKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
IEGRMDPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TVDKSRWQQGNVFSCSVMHEALHNHYTQKSLSLSPGK.

In some embodiments, the polypeptide is a mutant of PD-L2 with disabled inhibitory site. In some embodiments, the polypeptide includes an amino acid sequence which is at least 60%, 62%, 65%, 67%, 70%, 72%, 75%, 77%, 80%, 82%, 85%, 87%, 90%, 92%, 95%, 97%, 98%, or 99% identical to SEQ ID NO: 3. In some embodiments, the polypeptide includes an amino acid sequence which is at least 60%, 62%, 65%, 67%, 70%, 72%, 75%, 77%, 80%, 82%, 85%, 87%, 90%, 92%, 95%, 97%, 98%, or 99% identical to SEQ ID NO: 4. In some embodiments, the polypeptide includes an amino acid sequence which is at least 60%, 65%, 70%, 75%, 80%, 85%, 90%, 95%, or 99% identical to SEQ ID NO: 3. In some embodiments, the polypeptide includes an amino acid sequence which is at least 60%, 65%, 70%, 75%, 80%, 85%, 90%, 95%, or 99% identical to SEQ ID NO: 4. In some embodiment, the polypeptide includes an amino acid sequence which is SEQ ID: 3. In some embodiment, the polypeptide includes an amino acid sequence which is SEQ ID: 4. In some embodiment, the polypeptide includes one or more amino acids each selected from the group consisting of Tyr 112, Trp 110, Ile 103, Ile 105, Gln 101, and Tyr 114.

In some embodiments, the polypeptide binds to PD-1. In some embodiments, the polypeptide binds to PD-1 only through a binding site similar to the activating site of PD-L2. In some embodiments, the polypeptide does not have a binding site similar to the inhibitory site of the PD-L2. In some embodiments, the polypeptide activates PD-1. In some embodiments, the polypeptide activates immune cells through activating PD-1. Non-limiting examples of immune cells include T cells (e.g., Tregs), B cells, macrophages, and glial cells (e.g., astrocytes, microglia, or oligodendrocytes). In some embodiments, the polypeptide can be used to induce, promote, or enhance an immune response. In some embodiments, the polypeptide can be used to treat cancer.

SEQ ID NO: 5 is the amino acid sequence defining a third mutant of PD-L2:

LFTVTVPKELYIIEHGSNVTLECNFDTGSHVNLGAITASLQKVENDTSPH
RERATLLEEQLPLGKASFHIPQVQVRDEGQYLCDIIYGVAWDDKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
IEGRMDPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TVDKSRWQQGNVFSCSVMHEALHNHYTQKSLSLSPGK.

SEQ ID NO: 6 is the amino acid sequence defining a fourth mutant of PD-L2:

LFTVTVPKELYIIEHGSNVTLECNFDTGSHVNLGAITASLQKVENDTSPH
RERATLLEEQLPLGKASFHIPQVQVRDEGQYLCDIDYGVADDDKDLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
IEGRMDPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TVDKSRWQQGNVFSCSVMHEALHNHYTQKSLSLSPGK.

In some embodiments, the polypeptide is a mutant of PD-L2 with disabled activating site. In some embodiments, the polypeptide includes an amino acid sequence which is at least 60%, 62%, 65%, 67%, 70%, 72%, 75%, 77%, 80%, 82%, 85%, 87%, 90%, 92%, 95%, 97%, 98%, or 99% identical to SEQ ID NO: 5. In some embodiments, the polypeptide includes an amino acid sequence which is at least 60%, 62%, 65%, 67%, 70%, 72%, 75%, 77%, 80%, 82%, 85%, 87%, 90%, 92%, 95%, 97%, 98%, or 99% identical to SEQ ID NO: 6. In some embodiments, the polypeptide includes an amino acid sequence which is at least 60%, 65%, 70%, 75%, 80%, 85%, 90%, 95%, or 99% identical to SEQ ID NO: 5. In some embodiments, the polypeptide includes an amino acid sequence which is at least 60%, 65%, 70%, 75%, 80%, 85%, 90%, 95%, or 99% identical to SEQ ID NO: 6. In some embodiment, the polypeptide includes an amino acid sequence which is SEQ ID: 5. In some embodiment, the polypeptide includes an amino acid sequence which is SEQ ID: 6. In some embodiment, the polypeptide includes one or more amino acids each selected from the group consisting of Ile 105, Val 108, Gly 107, Ala 109, Trp 110, and Asp 111.

In some embodiments, the polypeptide binds to PD-1. In some embodiments, the polypeptide binds to PD-1 only through a binding site similar to the inhibitory site of PD-L2. In some embodiments, the polypeptide does not have a binding site similar to the activating site of PD-L2. In some embodiments, the polypeptide inhibits PD-1. In some embodiments, the polypeptide suppresses immune cells through inhibiting PD-1. Non-limiting examples of immune cells include T cells (e.g., Tregs), B cells, macrophages, and glial cells (e.g., astrocytes, microglia, or oligodendrocytes). In some embodiments, the polypeptide can be used to reduce, suppress, or prevent an immune response. In some embodiments, the polypeptide can be used to treat an autoimmune disease.

SEQ ID NO: 7 is the amino acid sequence defining a fifth mutant of PD-L2 on the linker region:

IDTVTVPKELYIIEHGSNVTLECNFDTGSHVNLGAITASLQKVENDTSPH
RERATLLEEQLPLGKASFHIPQVQVRDEGQYQCIIIYGVDWDYKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
IEGRMDPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TVDKSRWQQGNVFSCSVMHEALHNHYTQKSLSLSPGK.

In some embodiments, the polypeptide includes an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7.

In some embodiments, a polypeptide blocking or disabling the inhibitory site of PD-L2 is described, which activates PD-1 and immune response. For example, as illustrated in FIG. 2B, a polypeptide that blocks the activating site of PD-L2 can induce an inhibitory signal from PD-1. Further, as illustrated in FIG. 2C, a polypeptide that blocks the inhibitory site of PD-L2 can induce an activating signal from PD-1.

SEQ ID NO: 11 is the amino acid sequence defining a first polypeptide that binds to PD-L2:

TSESFVLNWYRMSPS.

SEQ ID NO: 12 is the amino acid sequence defining a second polypeptide that binds to PD-L2:

FSNTSESFVLNWYRM.

SEQ ID NO: 13 is the amino acid sequence defining a third polypeptide that binds to PD-L2:

ESFVLNWYRMSP.

In some embodiments, a polypeptide that blocks the inhibitory site of PD-L2 is described. In some embodiments, the polypeptide includes an amino acid sequence which is at least 60%, 65%, 70%, 75%, 80%, 85%, 90%, 95%, or 99% identical to SEQ ID NO: 11. In some embodiments, the polypeptide includes an amino acid sequence which is at least 60%, 65%, 70%, 75%, 80%, 85%, 90%, 95%, or 99% identical to SEQ ID NO: 12. In some embodiments, the polypeptide includes an amino acid sequence which is at least 60%, 65%, 70%, 75%, 80%, 85%, 90%, 95%, or 99% identical to SEQ ID NO: 13. In some embodiments, the polypeptide includes an amino acid sequence which is SEQ ID NO: 11. In some embodiments, the polypeptide includes an amino acid sequence which is SEQ ID NO: 12. In some embodiments, the polypeptide includes an amino acid sequence which is SEQ ID NO: 13.

In some embodiments, the polypeptide binds to PD-L2. In some embodiments, the polypeptide activates PD-1 through blocking the inhibitory site of PD-L2. In some embodiments, the polypeptide activates immune cells through activating PD-1. Non-limiting examples of immune cells include T cells (e.g., Tregs), B cells, macrophages, and glial cells (e.g., astrocytes, microglia, or oligodendrocytes). In some embodiments, the polypeptide can be used to induce, promote, or enhance an immune response. In some embodiments, the polypeptide can be used to treat cancer.

SEQ ID NO: 14 is the amino acid sequence defining a fourth polypeptide that binds to PD-L2:

TYLCGAISLAPKAQI.

In some embodiments, a polypeptide that blocks the activating site of PD-L2 is described. In some embodiments, the polypeptide includes an amino acid sequence which is at least 60%, 65%, 70%, 75%, 80%, 85%, 90%, 95%, or 99% identical to SEQ ID NO: 14. In some embodiments, the polypeptide includes an amino acid sequence which is SEQ ID NO: 14.

In some embodiments, the polypeptide binds to PD-L2. In some embodiments, the polypeptide inhibits PD-1 through blocking the activating site of PD-L2. In some embodiments, the polypeptide suppresses immune cells through inhibiting PD-1. Non-limiting examples of immune cells include T cells (e.g., Tregs), B cells, macrophages, and glial cells (e.g., astrocytes, microglia, or oligodendrocytes). In some embodiments, the polypeptide can be used to reduce, suppress, or prevent an immune response. In some embodiments, the polypeptide can be used to treat an autoimmune disease.

In some embodiments, the polypeptide can be modified by in ways known to one of ordinary skills in the art. For example, the polypeptides may be used as peptidomimetics. The polypeptides may form dimers.

In some embodiments, the polypeptides can be pegylated. Pegylation can delay the elimination of the polypeptides from the circulation by a variety of mechanisms. In some embodiments, pegylation inhibits degradation by proteolytic enzymes and, by increasing the apparent molecular size, reduces the rate of renal filtration. Accordingly, PEG-based modifications may be useful to prolong circulation time and bioavailability of the polypeptides. In some embodiments, the polypeptide is pegylated with linear PEG molecules. In another embodiment, the polypeptide is pegylated with branched PEG molecules. The invention further provides amino-, carboxy- and side chain-pegylated polypeptides. The PEG moiety can be a PEG molecule with a molecular weight greater than 5 kDa. For example the molecular weight can be between 5 kDa and 100 kDa (e.g., 5, 10, 15, 20, 25, 30, 35, 40, 50, 55, 60, 65, 70, 75, 80, 85, 90, 95 or 100 kDa), and more preferably a molecular weight of between 10 kDa and 50 kDa (e.g., 10, 12, 14, 16, 18, 20, 22, 24, 26, 28, 30, 32, 34, 36, 38, 40, 42, 44, 46, 48, or 50 kDa). Methods for synthesis of pegylated polypeptides are well known in the art.

In some embodiments, the polypeptide is attached to a detectable marker. In one embodiment, the detectable marker is attached at the C-terminus of the polypeptide. In another embodiment, the detectable label is attached to the N-terminus. A detectable marker can be a chemical label such as, but not limited to, radioactive isotopes, fluorescent groups, chemiluminescent labels, colorimetric labels, enzymatic markers, and affinity moieties (e.g., biotin) that facilitate detection of the labeled polypeptide. The invention also provides dye-labeled polypeptides such as, but not limited to, fluorescein and rhodamine conjugates. Other chemical labels and methods for attaching chemical labels to polypeptides are well-known in the art.

In some embodiments, the polypeptide is attached to a carrier molecule. The polypeptide may also be used as a conjugate of at least one polypeptide or a polypeptide fragment bound to a carrier. The carrier can provide solid phase support for the polypeptide of the invention. The carrier may be a biological carrier such as, but not limited to, a glycosaminoglycan, a proteoglycan, or albumin, or it may be a synthetic polymer such as, but not limited to a polyalkyleneglycol or a synthetic chromatography support. Other carriers include, but are not limited to, ovalbumin and human serum albumin, other proteins, and polyethylene glycol.

In one aspect, a nucleic acid encoding the polypeptides is described. In another embodiment, the polypeptides can be prepared using recombinant DNA technology methods, where an expression vector includes a nucleic acid sequence encoding the polypeptides of the invention, and where the nucleic acid sequence is operably linked to a promoter. The expression vector can be delivered by, for example, but not limited to, methods of transformation, transfection, etc., and a suitable host cell that allows expression of the polypeptide. Host cells including the expression vector are cultured under appropriate conditions and the polypeptide is expressed. In one embodiment, the host cell is a mammalian cell, including, but not limited to, a human cell. In another embodiment, the host cell is a bacterial, fungal or insect cell. In one embodiment, the polypeptide is recovered from the culture, where the recovery may include a step that leads to the purification of the polypeptide. Preparation of the polypeptides by recombinant technology can be advantageous if the polypeptides can be post-translationally modified. Further still, a combination of synthesis and recombinant DNA techniques can be employed to produce amide and ester derivatives of the polypeptides, as well as to produce fragments of the desired polypeptide which are then assembled by methods well known to those skilled in the art.

Expression vectors suitable for nucleic acid sequence delivery and polypeptide expression in human cells are known in the art. Non-limiting examples are plasmid, viral or bacterial vectors.

Polypeptides may also be prepared commercially by companies providing polypeptide synthesis as a service (e.g., BACHEM Bioscience, Inc., King of Prussia, Pennsylvania; AnaSpec, Inc., San Jose, California). Automated polypeptide synthesis machines, such as those manufactured by Perkin-Elmer Applied Biosystems, also are available.

The polypeptides useful in the methods described herein are purified once they have been isolated or synthesized by either chemical or recombinant techniques. Standard methods for purification purposes can be used, including reversed-phase high-pressure liquid chromatography (“HPLC”) using an alkylated silica column, such as, but not limited to, C⁴-, C₂- or C₁₈-silica. In this method, a gradient mobile phase of increasing organic content is generally used to achieve purification (e.g., acetonitrile in an aqueous buffer, usually containing a small amount of trifluoroacetic acid). Alternatively, ion-exchange chromatography can also be used to separate polypeptide compounds based on their charge. The degree of purity of the polypeptide compound may be diagnosed by the number of peaks identified by HPLC. In some embodiments, a useful level of polypeptide purity can result in a single peak on the HPLC chromatogram. In one embodiment, the polypeptide of interest is at least 94.99% of the input material on the HPLC column. In another embodiment, the polypeptide of interest is at least 96.99% of the input material on the HPLC column. In one embodiment, the polypeptide of interest is between 97% and 99.5% of the input material on the HPLC column.

In some embodiments, a method to determine whether or not a polypeptide exhibits an effect on immune response is the luciferase assay. In some embodiments, Jurkat PD-1 cells are used in luciferase assays.

### Pharmaceutical Compositions

In one aspect, a pharmaceutical composition is described, including one or more polypeptides as disclosed herein. In some embodiments, the polypeptide is encapsulated in a liposome in the pharmaceutical composition.

In some embodiments, carriers may be used in the pharmaceutical compositions, including, but not limited to, ion exchangers, alumina, aluminum stearate, lecithin, non-albumin serum proteins, buffer substances (e.g., phosphates, glycine, sorbic acid, potassium sorbate), and partial glyceride mixtures of saturated vegetable fatty acids, water, salts or electrolytes (e.g., protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat). Such modifications may both increase the apparent affinity and change the stability of a polypeptide. Although the number of polypeptide fragments bound to each carrier can vary, typically about 4 to 8 polypeptide fragments per carrier molecule are bound under standard coupling conditions.

In some embodiments, the polypeptides may also be prepared and stored in a salt form. Various salt forms of the polypeptides may also be formed or interchanged by any of the various methods known in the art, e.g., by using various ion exchange chromatography methods. Cationic counter ions that may be used in the compositions include, but are not limited to, amines (e.g., ammonium ions), metal ions, especially monovalent, divalent, or trivalent ions of alkali metals (e.g., sodium, potassium, lithium, and cesium); alkaline earth metals (e.g., calcium, magnesium, and barium); transition metals (e.g., iron, manganese, zinc, cadmium, and molybdenum); other metals like aluminum; and possible combinations of these. Anionic counter ions that may be used in the compositions described herein include, but are not limited to, chloride, fluoride, acetate, trifluoroacetate, phosphate, sulfate, carbonate, citrate, ascorbate, sorbate, glutarate, ketoglutarate, and possible combinations of these. Trifluoroacetate salts of polypeptide compounds described herein are typically formed during purification in trifluoroacetic acid buffers using HPLC. Although usually not suited for in vivo use, trifluoroacetate salt forms of the polypeptides described herein may be conveniently used in various in vitro cell culture studies, assays, or tests of activity or efficacy of a polypeptide compound of interest. The polypeptide may then be converted from the trifluoroacetate salt by ion exchange methods or synthesized as a salt form that is acceptable for pharmaceutical or dietary supplement compositions.

In some embodiments, the pharmaceutical composition can be delivered by a variety of routes or modes. These include, but are not limited to, parenteral, oral, intratracheal, sublingual, pulmonary, topical, rectal, nasal, buccal, sublingual, vaginal, or via an implanted reservoir. Implanted reservoirs may function by mechanical, osmotic, or other means. The term “parenteral” as used herein, includes, but is not limited to, intravenous, intracranial, intraperitoneal, paravertebral, periarticular, periosteal, subcutaneous, intracutaneous, intra-arterial, intramuscular, intra-articular, intrasynovial, intrasternal, intrathecal, and intralesional injection or infusion techniques. Such compositions are formulated for parenteral administration, and most for intravenous, intracranial, or intra-arterial administration. Generally, when administration is intravenous or intra-arterial, pharmaceutical compositions may be given as a bolus, as separated doses.

In some embodiments, the pharmaceutical composition may be in the form of a sterile injectable preparation, for example, as a sterile injectable aqueous suspension. This suspension may be formulated according to techniques known in the art using suitable dispersing or suspending agents. The sterile injectable preparation may also be a sterile injectable solution or suspension in a non-toxic parenterally acceptable diluent or solvent, for example, as a solution in 1,3-butanediol. Acceptable solvents that may be employed include, but are not limited to, mannitol, water, Ringer's solution, and isotonic sodium chloride solution. In addition, sterile, fixed oils are conventionally employed as a solvent or suspending medium. For this purpose, any bland fixed oil may be employed including, but not limited to, synthetic mono- or diglycerides. Fatty acids, such as oleic acid and its glyceride derivatives are useful in the preparation of injectables, as are natural pharmaceutically-acceptable oils, such as olive oil or castor oil.

In some embodiments, the pharmaceutical composition may be orally administered via capsules, tablets, caplets, pills, aqueous suspensions and solutions, or syrups. In the case of tablets for oral use, carriers, including, but not limited to, lactose and cornstarch, may be used. Lubricating agents, such as, but not limited to, magnesium stearate, are also sometimes added. For oral administration in a capsule form, useful diluents include, but are not limited to, lactose and dried cornstarch. Capsules, tablets, pills, and caplets may be formulated for delayed or sustained release when long-term expression is required. Alternatively, when oral aqueous suspensions are to be administered, the polypeptide is advantageously combined with emulsifying and/or suspending agents. If desired, certain sweetening and/or flavoring and/or coloring agents may be added. In one embodiment, the preparation for oral administration provides a polypeptide as described herein in a mixture that prevents or inhibits hydrolysis of the polypeptide compound by the digestive system, thereby allowing absorption into the blood stream.

In some embodiments, the pharmaceutical composition may be administered mucosally (e.g., vaginally or rectally). These dosages can be prepared by mixing a polypeptide as described herein with a suitable non-irritating excipient, which is solid at room temperature but liquid at body temperature and therefore will change states to liquid form in the relevant body space to release the active compound. Examples of these solvents include, but are not limited to, cocoa butter, beeswax, and polyethylene glycols. Still, for other mucosal sites, such as for nasal or pulmonary delivery, absorption may occur via the mucus membranes of the nose, or inhalation into the lungs. These modes of administration typically require that the composition be provided in the form of a solution, liquid suspension, or powder, which is then mixed with a gas such as air, oxygen, or nitrogen, or combinations thereof, so as to generate an aerosol or suspension of droplets or particles. These preparations are carried out according to well-known techniques in the art of pharmaceutical formulation. These preparations may be made as solutions in saline, employing benzyl alcohol or other suitable preservatives, absorption promoters to enhance bioavailability, fluorocarbons, and solubilizing or dispersing agents known in the art.

In some embodiments, the pharmaceutical composition further includes one or more additional therapeutic agents.

Exemplary additional therapeutic agents include, but are not limited to, cytokines, chemotherapeutic agents, radionuclides, other immunotherapeutics, enzymes, antibiotics, antivirals (e.g., protease inhibitors alone or in combination with nucleosides for treatment of HIV or hepatitis B or C), anti-parasites (e.g., helminths or protozoans), growth factors, growth inhibitors, hormones, hormone antagonists, antibodies and bioactive fragments thereof (e.g., humanized, single-chain, and chimeric antibodies), antigen and vaccine formulations (including adjuvants), polypeptide drugs, anti-inflammatories, ligands that bind to Toll-like receptors (including, but not limited to, CpG oligonucleotides) to activate the innate immune system, molecules that mobilize and optimize the adaptive immune system, other molecules that activate or up-regulate the action of cytotoxic T lymphocytes, NK cells and helper T cells, and other molecules that deactivate or down-regulate suppressor or regulatory T cells.

The additional therapeutic agents are selected based on the condition, disorder, or disease to be treated. For example, the polypeptides described herein can be co-administered with one or more additional agents that function to enhance or promote an immune response or reduce or inhibit an immune response.

### Chemotherapeutic Agents

In some embodiments, the polypeptides described herein can be combined with one or more chemotherapeutic agents or pro-apoptotic agents. Representative chemotherapeutic agents include, but are not limited to, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, and combinations thereof. Representative pro-apoptotic agents include, but are not limited to fludarabinetaurosporine, cycloheximide, actinomycin D, lactosylceramide, 15d-PGJ(2), and combinations thereof.

### Immunosuppressive Agents

In some embodiments, the polypeptide disclosed herein is used in combination with other immune therapeutic agents, immune modulators, costimulatory activating agonists, other cytokines and chemokines and factors, vaccines, oncolytic viruses, cell therapy, small molecules and targeted therapy, chemotherapy, and radiation therapy. In some embodiments, the immune modulators include check point inhibitors such as, but not limited to, anti-PD1, anti-CTLA4, anti-TIM3, and anti-LAG3. In some embodiments, the costimulatory activating agonists include anti-OX40, anti-GITR, and the like. In some embodiments, the cell therapy includes engineered T cells, CAR-T, TCR-T cells, and others.

In some embodiments, the polypeptides disclosed herein are used in combination with other immune therapeutic agents, immune modulators, biologics (e.g., antibodies), vaccines, small molecules and targeted therapy, anti-inflammatory, cell therapy (e.g., engineered Tregs and other type of cells), chemotherapy, and radiation therapy.

In some embodiments, the polypeptides disclosed herein, either used alone or in combination with other agents, are administered in vivo to a patient by intravenous, intramuscular, or other parenteral means. They can also be administered by intranasal application, inhalation, rectally, vaginally, topically, orally, or as implants.

In some embodiments, the additional therapeutic agent is an immune suppressant. Immunosuppressive agents include, but are not limited to, antibodies against other lymphocyte surface markers (e.g., CD40, and alpha-4 integrin) or against cytokines, fusion proteins (e.g., CTLA-4-Ig (Orencia®), and TNFR-Ig (Enbrel®)), TNF-α blockers, such as Enbrel, Remicade, Cimzia, and Humira, cyclophosphamide (“CTX”) (e.g., Endoxan®, Cytoxan®, Neosar®, Procytox®, and Revimmune™), methotrexate (“MTX”) (e.g., Rheumatrex® and Trexall®), belimumab (e.g., Benlysta®), other immunosuppressive drugs (e.g., cyclosporin A, FK506-like compounds, rapamycin compounds, and steroids), anti-proliferatives, cytotoxic agents, and other compounds that may assist in immunosuppression.

In some embodiments, the additional therapeutic agent can be a checkpoint inhibitor. In some embodiments, the additional therapeutic agent can be a CTLA-4 fusion protein, such as CTLA-4-Ig (abatacept). CTLA-4-Ig fusion proteins can compete with the co-stimulatory receptor, CD28, on T-cells for binding to CD80/CD86 (B7-1/B7-2) on antigen presenting cells, and thus function to inhibit T cell activation. In another embodiment, the additional therapeutic agent is a CTLA-4-Ig fusion protein known as belatacept. Belatacept contains two amino acid substitutions (L104E and A29Y) that can markedly increase its avidity to CD86 in vivo. In another embodiment, the additional therapeutic agent is Maxy-4.

In another embodiment, the additional therapeutic agent is CTX. CTX (the generic name for Endoxan®, Cytoxan®, Neosar®, Procytox®, and Revimmune™), also known as cytophosphane, is a nitrogen mustard alkylating agent from the oxazophorines group. It can be used to treat various types of cancer and some autoimmune disorders. CTX is the primary drug used for diffuse proliferative glomerulonephritis in patients with renal lupus.

In some embodiments, the additional therapeutic agent can be administered in an effective amount to reduce the blood or serum levels of anti-double-stranded DNA (“anti-ds DNA”) auto antibodies and/or to reduce proteinuria in a patient in need thereof.

In another embodiment, the additional therapeutic agent can increase the amount of adenosine in the serum (see, for example, WO 08/147482). For example, the second therapeutic agent can be CD73-Ig, recombinant CD73, or another agent (e.g., a cytokine, monoclonal antibody, or small molecule) that increases the expression of CD73 (see, for example WO 04/084933). In another embodiment, the additional therapeutic agent is Interferon-beta.

In some embodiments, the additional therapeutic agent can be a small molecule that inhibits or reduces differentiation, proliferation, activity, cytokine production, and/or cytokine secretion by Th1, Th17, Th22, and/or other cells that secrete, or cause other cells to secrete, inflammatory molecules, including, but not limited to, IL-1β, TNF-α, TGF-beta, IFN-γ, IL-18 IL-17, IL-6, IL-23, IL-22, IL-21, and MMPs. In another embodiment, the additional therapeutic agent is a small molecule that interacts with Tregs, enhances Treg activity, promotes or enhances IL-10 secretion by Tregs, increases the number of Tregs, increases the suppressive capacity of Tregs, or combinations thereof.

In some embodiments, the additional therapeutic agent is an antibody, for example, a function-blocking antibody against a proinflammatory molecule such as IL-6, IL-23, IL-22, or IL-21.

In some embodiments, the additional therapeutic agent includes a nucleic acid. In some embodiments, the additional therapeutic agent includes a ribonucleic acid.

### Methods of Treating Disease

In another aspect, a method of treating a disease in a subject in need thereof includes administering to the subject an effective amount of a polypeptide as described herein.

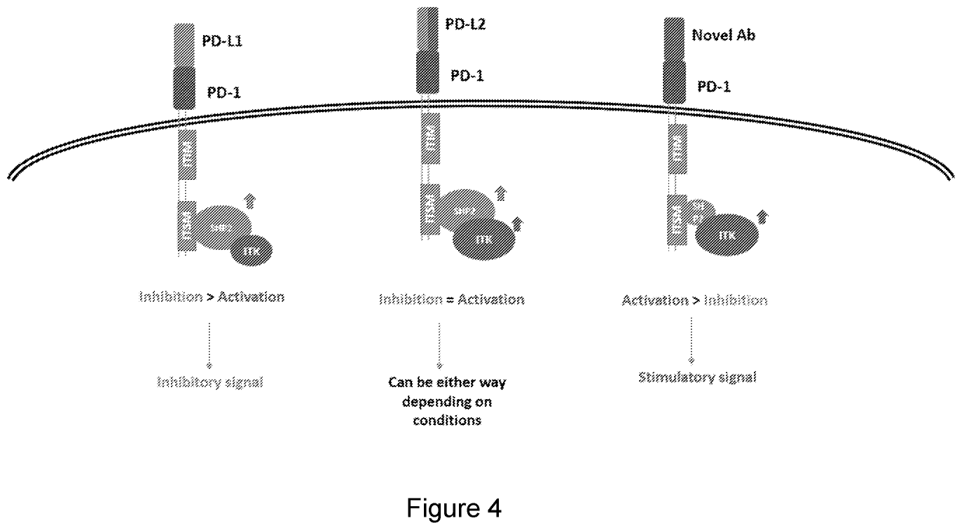

ITK plays an important role in immune response. The type of PD-1 signal is dependent on the balance of ITK and SHP2. In some embodiments, as illustrated in FIG. 4, PD-1 signal type depends on ITK-SHP2 ratio. In some embodiments, as illustrated in FIG. 4, when there is more SHP2 than ITK, an inhibitory signal is generated. In some embodiments, when there is more ITK than SHP 2, a stimulatory signal is generated. In some embodiments, an activator of ITK useful for treating cancer is described. In other embodiments, an inhibitor of ITK useful for treating autoimmune diseases is described. In some embodiments, the activator of ITK is a small-molecule compound, a polypeptide, or a nucleic acid. In some embodiments, the inhibitor of ITK is a small-molecule compound, a polypeptide, or a nucleic acid.

In another aspect, a method of treating a disease in a subject in need thereof includes administering to the subject an effective amount of an activator or inhibitor of ITK as described herein.

In some embodiments, the disease is cancer or an autoimmune disease.

In some embodiments, the polypeptide modulates PD-1 in immune cells. Non-limiting examples of immune cells include T cells (e.g., Tregs), B cells, macrophages, and glial cells (e.g., astrocytes, microglia, or oligodendrocytes). In some embodiments, the immune cells are Tregs. In some embodiments, the polypeptide activates PD-1 signaling. In other embodiments, the polypeptide inhibits PD-1 signaling. The inventors surprisingly found that, in some embodiments, the polypeptide activates immune response while, in other embodiments, the polypeptide suppresses immune response.

In some embodiments, activators of ITK can induce, promote, or enhance an immune response in a subject in need thereof. In other embodiments, inhibitors of ITK can reduce, suppress, or prevent an immune response in a subject in need thereof. In some embodiments, the activator of ITK is a small-molecule compound, a polypeptide, or a nucleic acid. In some embodiments, the inhibitor of ITK is a small-molecule compound, a polypeptide, or a nucleic acid.

In some embodiments, small molecules that bind to the inhibitory site of PD-1 is also described. In some embodiments, a small molecule that binds to the inhibitory site of PD-1 can reduce, suppress, or prevent an immune response in a subject in need thereof. In some embodiments, a small molecule that binds to the inhibitory site of PD-1 can be useful to treat an autoimmune disease.

### Cancer

In some embodiments, a method of treating or preventing cancer in a subject in need thereof is provided, including modulating PD-1 signaling through administering to the subject an effective amount of a polypeptide as described herein. In some embodiments, a method of treating or preventing cancer in a subject in need thereof is provided, including administering to the subject an effective amount of an activator of ITK.

In some embodiments, the cancer is selected from the group consisting of bladder cancer, brain cancer, breast cancer, cervical cancer, colorectal cancer, esophageal cancer, kidney cancer, liver cancer, lung cancer, nasopharyngeal cancer, pancreatic cancer, prostate cancer, skin cancer, stomach cancer, uterine cancer, ovarian cancer, testicular cancer, adult T-cell leukemia/lymphoma, and combinations thereof.

In some embodiments, the polypeptides and compositions disclosed herein are useful for treating leukemia. In some embodiments, the polypeptides and compositions disclosed herein that activate PD-1 are useful for treating leukemia. In these embodiments, the polypeptides and compositions disclosed herein that activate PD-1 are useful in vivo and ex vivo as immune response-stimulating therapeutics. The ability to activate PD-1 enables a more robust immune response. In some embodiments, the polypeptides and compositions disclosed herein are also useful to stimulate or enhance immune-stimulating or -activating responses involving T cells. In some embodiments, the polypeptides and compositions disclosed herein are useful for stimulating or enhancing an immune response in a host for treating leukemia by selectively activating PD-1. In these embodiments, the polypeptides and compositions disclosed herein can be administered to a subject in an amount effective to stimulate T cells in the subject. The types of leukemia that can be treated with the polypeptides and compositions as disclosed herein include, but are not limited to, acute myeloid leukemia (“AML”), chronic myeloid leukemia (“CML”), acute lymphocytic leukemia (“ALL”), chronic lymphocytic leukemia (“CLL”), adult T-cell leukemia/lymphoma (“ATLL”), and chronic myelomonocytic leukemia (“CMML”).

In some embodiments, ATLL is almost exclusively diagnosed in adults, with a median age in the mid-60s. In some embodiments, there are four types of ATLL: (1) acute, (2) chronic, (3) smoldering, and (4) lymphomatous. In some embodiments, acute ATLL is the most common form, and is characterized by high white blood cell count, hypercalcemia, organomegaly, and high lactose dehydrogenase. In some embodiments, lymphomatous ATLL manifests in the lymph nodes with less than 1% circulating lymphocytes. In some embodiments, chronic and smoldering ATLL are characterized by a less aggressive clinical course and allow for long-term survival. In some embodiments, the four-year survival rate for acute and lymphomatous ATLL is less than 5%. In some embodiments, chronic and smoldering forms of ATLL have four-year survival rates of 26.9% and 62%, respectively. In some embodiments, the adult T-cell leukemia/lymphoma is caused by HTLV-1.

In some embodiments, the polypeptides and compositions disclosed herein are useful for treating ATLL. In some embodiments, the polypeptides and compositions disclosed herein that activate PD-1 are useful for treating ATLL. In some embodiments, ATLL cells display an activated helper/inducer T-cell phenotype but exhibit strong immunosuppressive activity. In some embodiments, the polypeptides and compositions disclosed herein that activate PD-1 reduce the immunosuppressive response of the ATLL cells. In other embodiments, the polypeptides and compositions disclosed herein that activate PD-1 increase an immune stimulatory response to overcome the strong immunosuppressive activity of ATLL cells.

### Autoimmune Disease

In some embodiments, a method of treating or preventing an autoimmune disease in a subject in need thereof is provided, including modulating PD-1 signaling through administering to the subject an effective amount of a polypeptide as described herein. In some embodiments, a method of treating or preventing an autoimmune disease in a subject in need thereof is provided, including administering to the subject an effective amount of an inhibitor of ITK.

Non-limiting examples of autoimmune disease include achalasia, Addison's disease, adult Still's disease, agammaglobulinemia, alopecia areata, amyloidosis, ankylosing spondylitis, anti-glomerular basement membrane disease, anti-tubular basement membrane antibody nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, autoimmune urticaria, axonal and neuronal neuropathy, Baló disease, Behcet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss syndrome, eosinophilic granulomatosis, cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura, pemphigoid gestationis, hidradenitis suppurativa (acne inversa), hypogammaglobulinemia, IgA nephropathy, IgG4-related sclerosing disease, immune thrombocytopenia purpura, inclusion body myositis, interstitial cystitis, juvenile arthritis, juvenile diabetes (type 1 diabetes), juvenile myositis, Kawasaki disease, Lambert-Eaton syndrome, leukocytoclastic vasculitis, lichen planus, lichen sclerosus, ligneous conjunctivitis, linear IgA disease, lupus, chronic Lyme disease, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, pediatric autoimmune neuropsychiatric disorder, paraneoplastic cerebellar degeneration, paroxysmal nocturnal hemoglobinuria, Parry Romberg syndrome, pars planitis (peripheral uveitis), Parsonage-Turner syndrome, pemphigus, peripheral neuropathy, perivenous encephalomyelitis, pernicious anemia, POEMS syndrome, polyarteritis nodosa, polyglandular syndrome type I, polyglandular syndrome type II, polyglandular syndrome type III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome, subacute bacterial endocarditis, Susac's syndrome, sympathetic ophthalmia, Takayasu's arteritis, temporal arteritis (giant cell arteritis), thrombocytopenia purpura, Tolosa-Hunt syndrome, transverse myelitis, ulcerative colitis, undifferentiated connective tissue disease, uveitis, vasculitis, vitiligo, Vogt-Koyanagi-Harada disease, and combinations thereof.

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

### Example 1. Biological Assays

Jurkat-PD-1-NFAT reporter cell line (BPS Bioscience) (“PD-1 reporter cells”) was cultured according to vendor's specifications. The PD-1 reporter cells were dispensed in a 384-well plate at 20,000 cells/well. Group 1 cells were not treated. Groups 2 and 3 cells were activated with Gibco™ Dynabeads™ Human T-Activator CD3/CD28 for T Cell Expansion and Activation (100:1 V/V cell: bead ratio). Before activation, Group 3 cells were pre-incubated with a polypeptide having an amino acid sequence of SEQ ID NO: 14 at an amount of 5 times excess for 15 minutes. All cells were incubated for 24 hours at 37° C. with 5% CO₂.

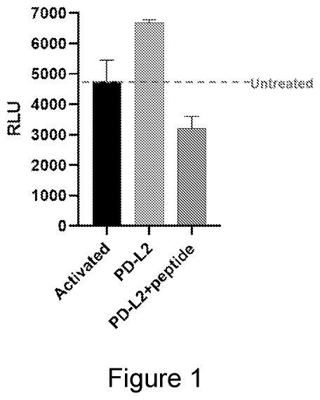

After 24 hours incubation, luciferase reagent (One-Step, Thermo Fisher Scientific) was added at 1:1 V/V ratio and incubated at room temperature for 15 minutes. Luminescence was measured at 100 ms setting on Varioscan Instrument (Thermo Fisher Scientific). The RLU of the three groups of cells are shown in FIG. 1.

### Example 2. Design of Polypeptide

FIGS. 2 and 3 show the design of polypeptides as described herein. FIG. 2A shows that PD-L2 has two interaction sites when binding to PD-1: one is the inhibitory site and the other is the activating site. FIG. 2B shows that when a polypeptide blocked the activating site of PD-L2, PD-L2 only bound to PD-1 through its inhibitory site, thereby inducing an inhibitory signal from PD-1. Further, as illustrated in FIG. 2C, for a polypeptide that blocked the inhibitory site of PD-L2, PD-L2 only bound to PD-1 through its activating site, thereby inducing an activating signal from PD-1.

FIG. 3 shows the design the PD-L2 mutants. As illustrated in FIG. 3A, a mutant of PD-L2 with a disabled activating site only bound to PD-1 through its inhibitory site. This binding to PD-1 could suppress immune response through PD-1 signal transduction. Furthermore, as illustrated in FIG. 3B, a mutant of PD-L2 with a disabled inhibitory site only bound to PD-1 through its activating site and therefore, activated immune response through PD-1 signal transduction.

### Example 3. Modulation of ITK

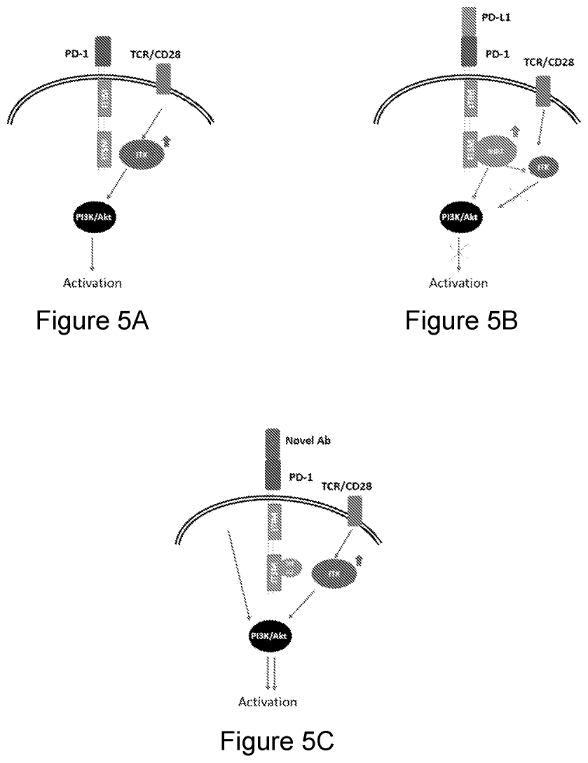

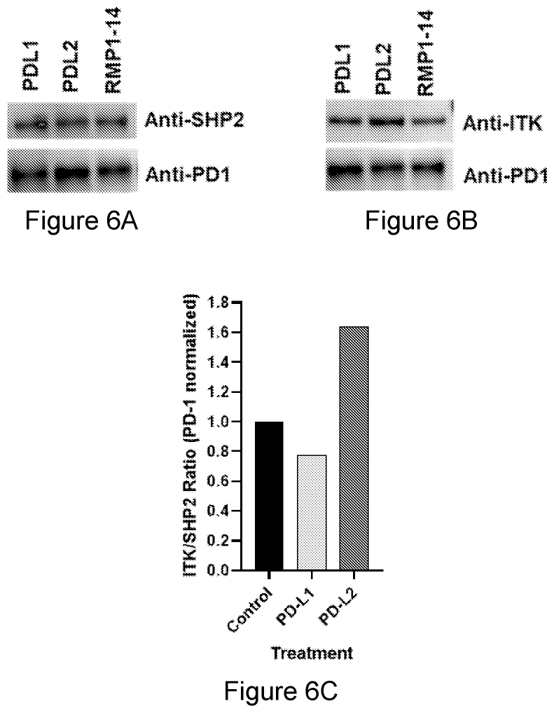

FIGS. 4 and 5 show the modulation of ITK and its effects on immune response. As illustrated in FIG. 4, when there is more SHP2 than ITK, an inhibitory signal was generated. However, when there is more ITK than SHP2, a stimulatory signal was generated. When there is the same amount of ITK and SHP2, the immune response was either inhibitory or activating. FIG. 5 shows that SHP2 is regulated by the upstream PD-1 pathway and ITK is affected by upstream T cell receptor (TCR) pathway. Thus, an activator of ITK would have the effect of enhancing an immune response and an inhibitor of ITK can suppress the immune response. FIG. 6 (T cells, activated for 48 hours, IP with Ab/ligand) further shows the levels of SHP2 and ITK that were bound to PD-1. PD-1 was immune-precipitated with beads 5 minutes after pre-activated primary mouse CD4 T cells were treated with PD-L1-IgG, PD-L2-IgG or anti-PD-1 mouse blocking antibody (clone RMP1-14). Western blot (“WB”) assay was used to determine levels of PD-1 bound ITK and SHP2 after different treatments, and showed increased ITK/SHP2 ratio after PD-L2 and slightly decreased ITK/SHP2 ratio after PD-L1 treatment compared to control Ab.

### Example 4. PD-L2 Increases Central Memory T Cells (“Tcm”) and Prevents T Cell Exhaustion

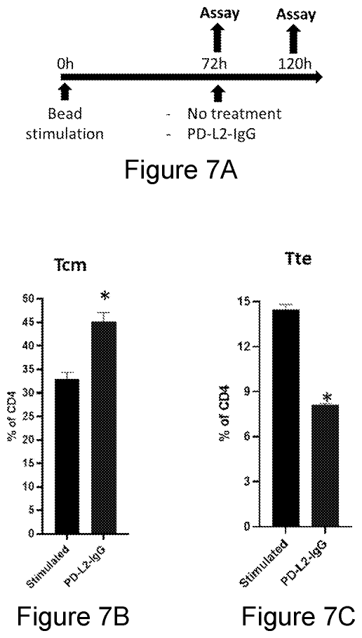

As illustrated in FIGS. 7A, flow cytometry sorted human CD4 T cells (BioIVT) were stimulated with Dyna beads (Gibco) (coated with anti-CD3 and anti-CD28 antibody) and IL-2 (100 U/mL—R&D Systems) for 72 hours. After 72 hours, the cells were treated with PD-L2-IgG at concentration of 25 μg/mL. Control wells were left untreated. Forty-eight hours after treatment, the cells were collected, washed and stained with the fluorophore labeled antibodies, and analyzed by flow cytometry. Tom were defined as CD45RO^{high}/CD62L^{high}/CD45RA^{low}. Exhausted T cells or terminal effector T cells (“Tte”) were defined as CD45RO^{low}/CD62L^{low}/CD45RA^{high}. As shown in FIGS. 7B-7C, PD-L2-IgG treatments led to a significant increase in Tcm (*p<0.05, FIG. 7B), and a significant decrease in Tte (*p<0.05, FIG. 7C).

### Example 5. PD-L2 is Required for Tcm Generation and Prevention of Exhaustion

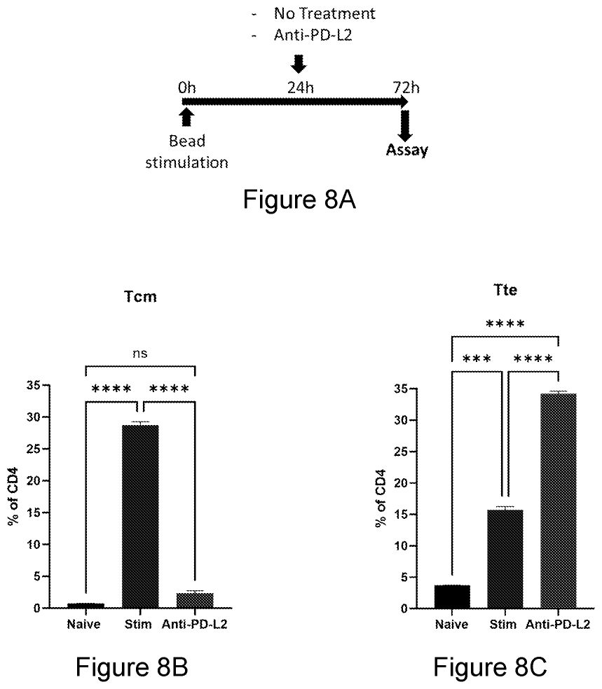

As illustrated in FIG. 8A, flow cytometry sorted human CD4 T cells (BioIVT) were stimulated with Dyna beads (Gibco) (coated with anti-CD3 and anti-CD28 antibody) and IL-2 (100 U/mL-R&D Systems) for 24 hours. After 24 hours, the cells were treated with anti-PD-L2 antibody at concentration of 100 μg/mL or left untreated. Forty-eight hours after treatment, the cells were collected, washed and stained with the fluorophore labeled antibodies, and analyzed by flow cytometry. Tcm were defined as CD45RO^{high}/CD62L^{high}/CD45RA^{low}. Tte were defined as CD45RO^{low}/CD62L^{low}/CD45RA^{high}. As shown in FIGS. 8B-8C, blocking the PD-L2 binding to PD-2 with anti-PD-L2 antibody prevented Tcm generation (**** p<0.0001, FIG. 8B) and significantly increased Tte (FIG. 8C).

### Example 6. PD-L2 is Required for Initiation of Activation of Both CD4 and CD8 T Cells

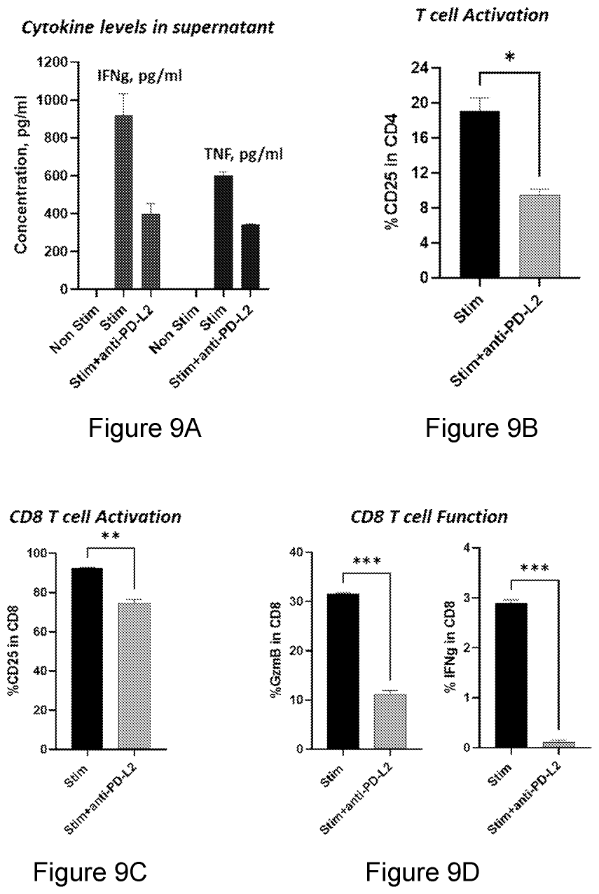

Flow cytometry sorted human CD4 and CD8 T cells (BioIVT) were stimulated with Dyna beads (Gibco) (coated with anti-CD3 and anti-CD28 antibody) and IL-2 (100 U/mL-R&D Systems) for 24 hours. After 24 hours, the cells were treated with anti-PD-L2 antibody at concentration of 100 μg/mL or left untreated. Forty-eight hours after treatment, the cells and supernatants were collected. The supernatants were analyzed for IFNg and TNF using CBA assay (BD Biosciences). The cells were washed and stained with the fluorophore labeled antibodies and analyzed by flow cytometry. CD25 expression on CD4 T cells, Granzyme B and IFNg in CD8 T cells was used as measure of activation and functionality. As shown in FIG. 9A, anti-PD-L2 treatment inhibited cytokine production for CD4 T cells, as well as decreased CD4 T cell activation (as shown in FIG. 9B). Similarly, PD-L2 blockade decreased CD25 expression on CD8 T cells (as shown in FIG. 9C), and decreased intracellular Granzyme B and IFNg (as shown in FIG. 9D).

1. An isolated polypeptide comprising an amino acid sequence which is at least 60%, 65%, 70%, 75%, 80%, 85%, 90%, 95%, or 99% identical to SEQ ID NO: 1.

2. An isolated polypeptide comprising an amino acid sequence which is SEQ ID NO: 21.

3. An isolated polypeptide comprising an amino acid sequence which is SEQ ID NO: 22.

4. The polypeptide of claim 1, wherein the amino acid is fused to an immunoglobulin.

5. The polypeptide of claim 4, wherein the immunoglobulin is IgG1, IgG2, IgG3, or IgG4.

6. The polypeptide of claim 1, wherein the polypeptide comprises an amino acid sequence which is at least 60%, 65%, 70%, 75%, 80%, 85%, 90%, 95%, or 99% identical to SEQ ID NO: 3 or SEQ ID NO: 4.

7. The polypeptide of claim 6, wherein the polypeptide comprises an amino acid sequence which is SEQ ID NO: 3 or SEQ ID NO: 4.

8. The polypeptide of claim 1, wherein the polypeptide comprises one or more amino acids each selected from the group consisting of Tyr 112, Trp 110, Ile 103, Ile 105, Gln 101, and Tyr 114.

9. The polypeptide of claim 1, wherein the polypeptide binds to programmed cell death protein 1 (“PD-1”) and activates PD-1.

10. The polypeptide of claim 1, wherein the polypeptide activates immune cells.

11. The polypeptide of claim 1, wherein the polypeptide comprises an amino acid sequence which is at least 60%, 65%, 70%, 75%, 80%, 85%, 90%, 95%, or 99% identical to SEQ ID NO: 5 or SEQ ID NO: 6.

12. The polypeptide of claim 11, wherein the polypeptide comprises an amino acid sequence which is SEQ ID NO: 5 or SEQ ID NO: 6.

13. The polypeptide of claim 1, wherein the polypeptide comprises one or more amino acids each selected from the group consisting of Ile 105, Val 108, Gly 107, Ala 109, Trp 110, and Asp 111.

14. The polypeptide of claim 1, wherein the polypeptide binds to PD-1 and inhibits PD-1.

15. The polypeptide of claim 1, wherein the polypeptide suppresses immune cells.

16. The polypeptide of claim 1, wherein the polypeptide induces central memory T cells (“Tcm”).

17. The polypeptide of claim 1, wherein the polypeptide prevents T cell exhaustion.

18-42. (canceled)

43. An isolated polypeptide comprising an amino acid sequence which is at least 60%, 65%, 70%, 75%, 80%, 85%, 90%, 95%, or 99% identical to any one of SEQ ID NOS: 11-13.

44. The polypeptide of claim 43, wherein the polypeptide comprises an amino acid sequence selected from the group consisting of SEQ ID NOS: 11-13.

45. The polypeptide of claim 43, wherein the polypeptide binds to programmed cell death 1 ligand 2 (“PD-L2”).

46. The polypeptide of claim 43, wherein the polypeptide blocks the inhibitory site of PD-L2.

47. The polypeptide of claim 43, wherein the polypeptide activates immune cells.

48. An isolated polypeptide comprising an amino acid sequence which is at least 60%, 65%, 70%, 75%, 80%, 85%, 90%, 95%, or 99% identical to SEQ ID NO: 14.

49. The polypeptide of claim 48, comprising an amino acid sequence which is SEQ ID NO: 14.

50. The polypeptide of claim 48, wherein the polypeptide binds to PD-L2.

51. The polypeptide of claim 48, wherein the polypeptide blocks the activating site of PD-L2.

52. The polypeptide of claim 48, wherein the polypeptide inhibits immune cells.

53-70. (canceled)

71. The polypeptide of claim 2, wherein the amino acid is fused to an immunoglobulin.

72. The polypeptide of claim 71, wherein the immunoglobulin is IgG1, IgG2, IgG3, or IgG4.

73. The polypeptide of claim 3, wherein the amino acid is fused to an immunoglobulin.

74. The polypeptide of claim 73, wherein the immunoglobulin is IgG1, IgG2, IgG3, or IgG4.

75. The polypeptide of claim 2, wherein the polypeptide comprises an amino acid sequence which is at least 60%, 65%, 70%, 75%, 80%, 85%, 90%, 95%, or 99% identical to SEQ ID NO: 3 or SEQ ID NO: 4.

76. The polypeptide of claim 75, wherein the polypeptide comprises an amino acid sequence which is SEQ ID NO: 3 or SEQ ID NO: 4.

77. The polypeptide of claim 2, wherein the polypeptide comprises one or more amino acids each selected from the group consisting of Tyr 112, Trp 110, Ile 103, Ile 105, Gln 101, and Tyr 114.

78. The polypeptide of claim 2, wherein the polypeptide binds to programmed cell death protein 1 (“PD-1”) and activates PD-1.

79. The polypeptide of claim 2, wherein the polypeptide activates immune cells.

80. The polypeptide of claim 3, wherein the polypeptide comprises an amino acid sequence which is at least 60%, 65%, 70%, 75%, 80%, 85%, 90%, 95%, or 99% identical to SEQ ID NO: 5 or SEQ ID NO: 6.

81. The polypeptide of claim 80, wherein the polypeptide comprises an amino acid sequence which is SEQ ID NO: 5 or SEQ ID NO: 6.

82. The polypeptide of claim 3, wherein the polypeptide comprises one or more amino acids each selected from the group consisting of Ile 105, Val 108, Gly 107, Ala 109, Trp 110, and Asp 111.

83. The polypeptide of claim 3, wherein the polypeptide binds to PD-1 and inhibits PD-1.

84. The polypeptide of claim 3, wherein the polypeptide suppresses immune cells.

85. The polypeptide of claim 3, wherein the polypeptide induces central memory T cells (“Tcm”).

86. The polypeptide of claim 3, wherein the polypeptide prevents T cell exhaustion.
